(19) United States
(12) Patent Application Publication
(10) Pub. No.: US 2026/0355508 A1
(43) Pub. Date: Nov. 12, 2026

(54) ELECTRODES AND TIP FOR DEVICE FOR ELECTRICAL STIMULATION

(71) Applicant: SEPTODONT, INC, Lancaster, PA (US)

(72) Inventor: Cosmo HARALAMBIDIS, Cranston, RI (US)

(21) Appl. No.: 19/673,812

(22) Filed: May 11, 2026

Related U.S. Application Data
(60) Provisional application No. 63/803,033, filed on May 9, 2025.

(51) Int. Cl. A61N 1/05; A61N 1/36

(52) U.S. Cl. CPC A61N 1/0548; A61N 1/36021

(57) ABSTRACT

Electrodes and a tip for a device for electrical stimulation include at least two electrodes of a rigid, electrically conductive material in a fixed spatial relationship. The at least two electrodes include at least a first electrode and a second electrode. The first electrode has a shape defining a first tip and a first rail. The second electrode has the shape defining a second tip and a second rail. In a first orientation, the first tip and the second tip can be applied to a bone, tooth, or tissue or other application area. In a second orientation, the first rail and the second rail can be applied to a bone, tooth, or tissue or other application area.

### CROSS-REFERENCE TO RELATED APPLICATIONS

The present application claims priority to, and the benefit of United States Provisional Application No. 63/803,033, filed May 9, 2025, entitled “Electrodes and Tip for Device for Electrical Stimulation” the contents of which are hereby incorporated by reference in their entirety.

### BACKGROUND

Electrical stimulation of bone and tissue has been shown to reduce pain, encourage tooth movement, and promote bone healing, especially in periodontal, dental, and orthodontic applications. Examples of devices and techniques are provided in U.S. Patents 9,855,418 and 11,969,590, which are hereby incorporated by reference.

### SUMMARY

This Summary introduces selected concepts in simplified form which are described further below in the Detailed Description. This Summary is intended neither to identify essential features, nor to limit the scope, of the claimed subject matter.

The electrical stimulation that is applied through such devices depends in part on the shape of the electrodes used on the device. It has been discovered that operators have more options for applying different strengths of electrical stimulation when the electrode has two or more ways to make contact with an application area such as bone, tooth, or tissue. For example, in addition to providing two or more points of contact such as through the respective tips of two or more electrodes, a pair of electrodes can have a shape enabling two longitudinal surfaces, or rails, one from each electrode, to be placed in contact with the bone, tooth, or tissue.

Accordingly, in one aspect electrodes for a device include at least a first electrode and a second electrode, wherein the first electrode has a shape defining a first point and a rail, and the second electrode has the shape defining a second point and a rail. In a first orientation, the first tip and the second tip can be applied to an application area, such as a bone, tooth, or tissue. In a second orientation, the first rail and the second rail can be applied to an application area, such as a bone, tooth, or tissue.

Accordingly, in one aspect a tip for a device includes at least a first electrode and a second electrode, wherein the first electrode has a shape defining a first tip and a first rail, and the second electrode has the shape defining a second tip and a second rail, such that, in a first orientation, the first tip and the second tip can be applied to a bone, tooth, or tissue, and in a second orientation, the first rail and the second rail can be applied to a bone, tooth, or tissue. The tip includes a base having a first mechanical connector and a first electrical connection, wherein the at least two electrodes are mounted in the base and are electrically connected to the first electrical connection.

In one aspect, a method of treatment includes using a device with such a tip, and applying the first tip and the second tip together to an application area, such as a bone, tooth, or tissue, and applying the first rail and the second rail together to an application area, such as a bone, tooth, or tissue.

In the following description, reference is made to the accompanying drawings which form a part hereof, and in which are shown, by way of illustration, specific example implementations of this technique. It is understood that other embodiments may be utilized, and structural changes may be made without departing from the scope of the disclosure.

### BRIEF DESCRIPTION OF THE DRAWINGS

FIG. 1 is a perspective view of a first example construction of a tip with electrodes.

FIG. 2 is a front view of the example construction of FIG. 1.

FIG. 3 is a left side view of the example construction of FIG. 1.

FIG. 4 is a top view of the example construction of FIG. 1.

FIG. 5 is a bottom view of the example construction of FIG. 1.

FIG. 6 is a back view of the example construction of FIG. 1.

FIG. 7 is a perspective view of a second example construction of a tip with electrodes.

### DETAILED DESCRIPTION

The following detailed description sets forth example constructions and configurations of electrodes on a tip of a device for electrical stimulation. The electrodes are made of a rigid, electrically conductive material, such as stainless steel or platinum, and are placed in a fixed spatial relationship on the tip for the device.

Such a device can be used to apply electrical stimulation to one or more components of the periodontal complex and surrounding tissue of a tooth, for uses such as reducing orthodontic pain and encouraging tooth movement, such as described in U.S. Patents 9,855,418 and 11,969,590, which are hereby incorporated by reference.

A device using such a tip with electrodes can be used by an operator to place electrodes on an application area. In some implementations, the electrodes are configured for application to oral mucosa and attached gingiva adjacent to, and along a periodontal ligament of, a root structure of a single tooth, or on the surface of a tooth, or on an area surrounding an implant to support a crown.

An electrical circuit (not shown) applies, through the electrodes, an electrical waveform. The frequency or frequencies, voltage, and current defining the electrical waveform, and duration of application, depend on the application. In some implementations, a subsensory or slightly sensory electrical stimulus can be applied.

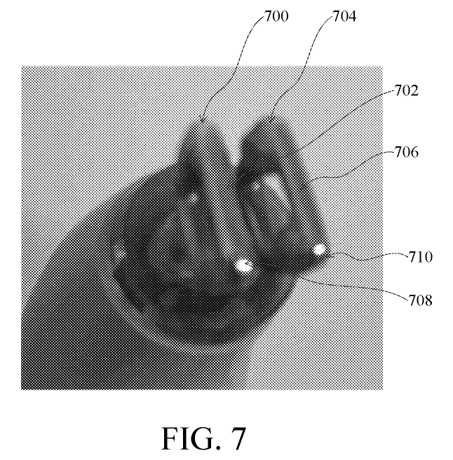

The first example construction of a tip with a pair of electrodes will now be described in connection with FIGS. 1-6. A second example construction is shown in FIG. 7.

FIG. 1 is a perspective view of a first example construction of a tip with electrodes. In FIG. 1, the first electrode has a shape defining a first tip 100 and a first rail 102. A second electrode has the same shape which defines a second tip 104 and a second rail 106. Each electrode also may have an additional rail e.g., 108, 110. In the example shown in FIG. 1, the shape is triangular. The invention is not limited to a triangular shape, and any shape of electrodes that provide both a pair of tip contacts and a pair of rail contacts can be used. The electrodes are formed in a base 112. The invention is not limited to a particular kind or shape of base. The base provides a mechanical and electrical connection to a device that provides the electrical waveform for performing electrical stimulation, and typically includes plastic, polycarbonate, ceramic or other insulating material encasing conductive material that connects the electrodes to an electrical connection.

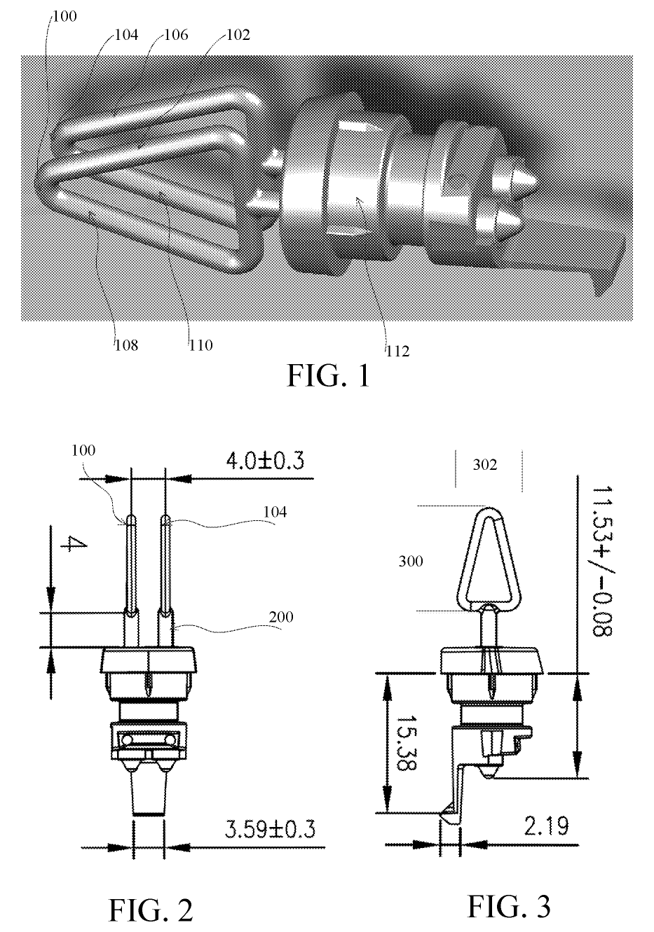

FIG. 2 is a front view of the example construction of FIG. 1. This view illustrates the fixed spatial relationship between the first electrode and the second electrode. In this example construction, at the first tip 100 and the second tip 104, the distance between the two tips is about four (4) millimeters. A similar distance is maintained between the two rails. In this example construction, the electrodes are elevated about the base on posts 200 at a height of about 4 millimeters. FIG. 6 is a back side view of the example construction of FIG. 1 and is similar to FIG. 2 except for the orientation of the base due to the symmetry of the electrodes.

FIG. 3 is a left side view of the example construction of FIG. 1. In this example construction, the first electrode and the second electrode have an identical shape. As an example only, a height 300 of the triangle can be about 14 millimeters and a width 302 of the triangle can be about 11 millimeters. A variety of dimensions can be used for different triangular shapes. An example thickness of each electrode is about 1.5 millimeters.

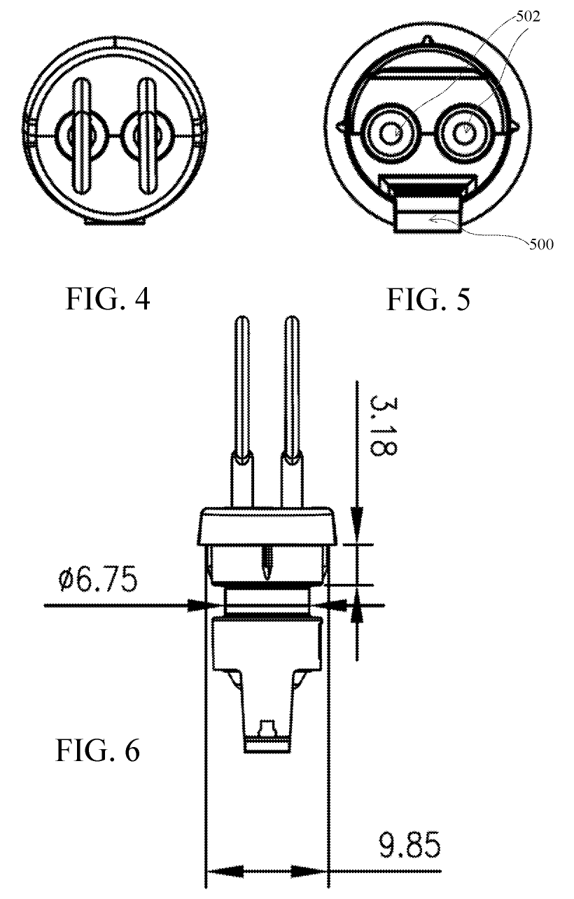

FIG. 4 is a top view of the example construction of FIG. 1, looking on a plane along the top of the base for the tip. While the electrodes are illustrated in FIGS. 2-4 with the tips and supporting posts aligned in a straight line perpendicular to the base, the electrodes can be angled.

FIG. 5 is a bottom view of the example construction of FIG. 1, which illustrates features of an example base. The invention is not limited to any particular base. The configuration of the base depends on the source of the electrical stimulation. Typically, the base has a mechanical connector 500 having a mating configuration with a mechanical connector for the device. The base also has an electrical connection 502 having a mating configuration with an electrical connection for the device. Such a construction of the housing and electrode interconnection allows for removal of the electrodes for cleaning or sterilization or for replacement. Typically, the base has a shape corresponding to the shape of a mating connector for the device.

FIG. 7 is a perspective view of a second example construction of a tip with electrodes. A first electrode has a shape defining a first tip 700 and a first rail 702. A second electrode has the same shape which defines a second tip 704 and a second rail 706. Each electrode also may have an additional tip e.g., 708, 710. In the example shown in FIG. 7, the shape also is triangular. The invention is not limited to a triangular shape, and any shape of electrodes that provide both a pair of tip contacts and a pair of rail contacts can be used. Similar to the other example constructions, the electrodes can be formed in a base.

While the Figures illustrate two electrodes it should be understood that at least two electrodes encompass more than two electrodes or probe contacts. There can be any number of contacts applied to an application area.

In some implementations, the spacing of the electrodes is such that they can span an adequate length along the periodontal ligament between the attached gingiva and the oral mucosa. For example, the spacing can be at least two millimeters. As another example, the spacing can be at least three millimeters. As another example, the spacing can be between two millimeters and six millimeters. As another example, the spacing can be between three and five millimeters. As another example, the spacing can be between 3.5 and 4.5 millimeters. As another example the spacing can be approximately four millimeters.

It should be understood that the subject matter defined in the appended claims is not necessarily limited to the specific implementations described above. The specific implementations described above are disclosed as examples only.

What is claimed is:

1. Electrodes for a device for electrical stimulation, comprising:
at least two electrodes of a rigid, electrically conductive material in a fixed spatial relationship, including at least a first electrode and a second electrode,
wherein the first electrode has a shape defining a first tip and a first rail, and
wherein the second electrode has the shape defining a second tip and a second rail.

2. The electrodes of claim 1, such that, in a first orientation, the first tip and the second tip can be applied to an application area, and in a second orientation, the first rail and the second rail can be applied to an application area.

3. The electrodes of claim 2, wherein the application area is a bone, tooth, or tissue.

4. A tip for a device for electrical stimulation, comprising:
at least two electrodes of a rigid, electrically conductive material in a fixed spatial relationship, including at least a first electrode and a second electrode,
wherein the first electrode has a shape defining a first tip and a first rail, and
wherein the second electrode has the shape defining a second tip and a second rail.
a base having a first mechanical connector and a first electrical connection, wherein the at least two electrodes are mounted in the base and are electrically connected to the first electrical connection.

5. The tip of claim 4, such that, in a first orientation, the first tip and the second tip can be applied to an application area, and in a second orientation, the first rail and the second rail can be applied to an application area.

6. The tip of claim 5, wherein the application area is a bone, tooth, or tissue.

7. A method of treatment using a device for electrical stimulation, comprising:
using a device with at least two electrodes
applying the first tip and the second tip together to an application area, and
applying the first rail and the second rail together to an application area.

8. The method of treatment of claim 7, wherein the application area is a bone, tooth, or tissue.
